(19) United States
(12) Patent Application Publication
(10) Pub. No.: US 2020/0261273 A1
(43) Pub. Date: Aug. 20, 2020

(54) METHOD FOR CONTROLLING AN EYE SURGICAL LASER AND TREATMENT DEVICE

(71) Applicant: SCHWIND EYE-TECH-SOLUTIONS GMBH, Kleinostheim (DE)

(72) Inventors: Samuel ARBA-MOSQUERA, Aschaffenburg (DE); Pacal NAUBEREIT, Aschaffenburg (DE); Thomas WENDLER, Stockstadt (DE)

(21) Appl. No.: 16/791,236

(22) Filed: Feb. 14, 2020

(30) Foreign Application Priority Data
Feb. 15, 2019 (DE) 10 2019 103 851.0

(51) Int. Cl. A61F 9/008

(52) U.S. Cl. CPC A61F 9/00836; A61F 2009/00872; A61F 9/0084

(57) ABSTRACT

The present invention relates to a method for controlling an eye surgical laser for the separation of a volume body with predefined interfaces from a human or animal cornea, comprising controlling the laser by means of a control device such that it emits pulsed laser pulses in a predefined pattern into the cornea, wherein the interfaces of the volume body to be separated are defined by the predefined pattern and the interfaces are generated by means of photodisruption, wherein the interfaces of the volume body are determined such that a pathological and/or unnaturally altered area within the stroma of the cornea is enclosed. Furthermore, the invention relates to a treatment device with at least one eye surgical laser for the separation of a predefined corneal volume with predefined interfaces of a human or animal eye by means of photodisruption and at least one control device for the laser or lasers, which is formed to execute the steps of the method according to the invention.

### FIELD

The present invention relates to a method for controlling an eye surgical laser for the separation of a volume body with predefined interfaces from a human or animal cornea. Furthermore, the invention relates to a treatment device with at least one eye surgical laser for the separation of a predefined corneal volume with predefined interfaces of a human or animal eye by means of photodisruption and at least one control device for the laser or lasers, to a computer program and a computer-readable medium, as well to a method for the separation of a volume body with predefined interfaces from a human or animal cornea.

### BACKGROUND

Opacities and scars within the cornea, which can arise by inflammations, injuries or native diseases, impair the sight. In particular in case that these pathological and/or unnaturally altered areas of the cornea are located in the axis of vision of the eye, clear sight is considerably disturbed. In known manner, the thus altered areas are eliminated by so-called phototherapeutic keratectomy (PTK) by means of an ablatively effective laser, for example an excimer laser. However, this is only possible if the pathological and/or unnaturally altered areas of the cornea are located in the superficial layers of the cornea. Subjacent areas, in particular within the stroma, are not reachable by means of ablative laser methods. Here, additional measures such as for example the exposure of the subjacent areas have to be taken by means of an additional corneal incision. By the additional measures, the treatment duration is disadvantageously considerably increased. In addition, there is the risk that further complications such as for example the occurrence of inflammations at the incision locations occur by the additional corneal incisions.

Therefore, it is the object of the present invention to provide a method and a treatment device for controlling an eye surgical laser for the separation of a volume body with predefined interfaces from a human or animal cornea, by which the disadvantages of the prior art are overcome.

### SUMMARY

A generic method according to the features of claim 1, a generic treatment device comprising the features of claim 9, a computer program according to the features of claim 12, a computer-readable medium according to the features of claim 13, and a method for the separation of a volume body with predefined interfaces from a human or animal cornea according to the features of claim 14 serve for solving this object.

Advantageous configurations with convenient developments of the invention are specified in the respective dependent claims, wherein advantageous configurations of the method are to be regarded as advantageous configurations of the treatment device, of the computer program and of the computer-readable medium and vice versa.

A first aspect of the invention relates to a method for controlling an eye surgical laser for the separation of a volume body with predefined interfaces from a human or animal cornea, wherein the method comprises controlling the laser by means of a control device such that it emits pulsed laser pulses in a predefined pattern into the cornea, wherein the interfaces of the volume body to be separated are defined by the predefined pattern and the interfaces are generated by means of the photodisruption. Therein, the interfaces of the volume body are determined such that a pathological and/or unnaturally altered area within the stroma of the cornea is enclosed. Therein, the laser is controlled such that at least one incision and/or at least one aperture is generated in the cornea at a predefined angle and with a predefined geometry, wherein the incision or the aperture intersects an interface of the volume body and is formed up to a surface of the cornea such that the volume body is removable from the cornea via the incision or the aperture and wherein the surface of the cornea is a surface of the eye artificially generated by means of ablation or displacement of an uppermost corneal layer and/or by means of production of a corneal flap. Thereby, the method according to the invention is usable for a plurality of phototherapeutic keratectomy methods. The possibilities of application are considerably increased.

By the method according to the invention, it is possible to reliably remove pathological and/or unnaturally altered areas in the stroma of the cornea that is in subjacent areas of the cornea. Additional exposure of these subjacent areas of the cornea by means of additional corneal incisions is basically not required. Thereby, the treatment duration can be considerably shortened, possible complications by the usually required additional corneal incisions are avoided.

Therein, the volume body can be lenticularly formed, whereby a simple removal via the mentioned incision or the mentioned aperture is possible. In that the volume body to be separated is only described and defined by the interfaces and these interfaces enclose the pathological and/or unnaturally altered tissue and the correspondingly altered area, respectively, on the one hand and are generated by means of photodisruption on the other hand, a full-area or full-volume ablation of the volume body can be omitted. Only the interfaces are generated by means of photodisruption such that the predefined volume body can subsequently be removed from the cornea. It is also to be understood by the term “interfaces” that the volume body can optionally be defined and separated by means of a single interface located in the cornea. By the method according to the invention, phototherapeutic keratectomy methods can be performed in deep areas of the cornea, in particular the stroma, on the one hand. On the other hand, the treatment duration for the separation of the volume body is shortened, the energy input into the cornea of the patient is additionally also significantly reduced. Therein, the lenticular volume body can be arranged in an area arranged concentrically around a center of the cornea or concentrically to an optical axis of the eye. However, it is also possible that the lenticular volume body is arranged in an area arranged non-concentrically around the center of the cornea or non-concentrically to the optical axis of the eye.

In further advantageous configurations of the method according to the invention, the laser is controlled such that the predefined pattern is at least partially circularly and/or spirally ablated. Therein, the start of the photodisruption can be effected by the individual laser pulses in the center of the respective interface or also at the edge of the respective interface.

In further advantageous configurations of the method according to the invention, the predefined pattern is defined based on one or more control datasets, wherein the control dataset or datasets include control data for positioning and/or for focusing individual laser pulses in the cornea. The determination of the control datasets is known and in particular results from the topographic and/or pachymetric measurement of the cornea to be treated as well as the type, the position and the extent of the pathologic and/or unnaturally altered area within the stroma of the cornea. In particular, the control datasets are generated at least by providing topographic and/or pachymetric and/or morphologic data of the untreated cornea and providing topographic and/or pachymetric and/or morphologic data of the pathologically and/or unnaturally altered area to be removed within the cornea.

In further advantageous configurations of the method according to the invention, the volume body to be separated is formed such that a refractive correction of the eye is additionally effected by the removal of the volume body. The mentioned visual disorders of the eye can be myopia, hyperopia, presbyopia, astigmatism or also other visual disorders of the eye. Furthermore, there is the possibility that the pathologically altered area within the cornea is an opacity and/or a scar.

In further advantageous configurations of the method according to the invention, the control device is formed such that the laser emits laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz. Such lasers are already used for photodisruptive methods in the eye surgery. Thus, EP 2 211 803 B1 for example describes a corresponding femto-second laser, which is used for producing a so-called lenticule, that is a lens-like volume body, within the cornea. The thus produced lenticule is subsequently removed from the cornea via an incision in it. However, the use of such photodisruptive lasers instead of ablatively effective lasers in the phototherapeutic keratectomy (PTK) is new and not known from the prior art. The use of photodisruptive lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive lasers of the type used here usually input pulsed laser radiation with a pulse duration between 1 fs and 1 ns into the corneal tissue. Thereby, the power density of the respective laser pulse required for the optical breakthrough can be spatially narrowly limited such that high cutting precision in generating the interfaces is ensured.

A second aspect of the present invention relates to a treatment device with at least one eye surgical laser for the separation of a predefined corneal volume with predefined interfaces of a human or animal eye by means of photodisruption and at least one control device for the laser or the lasers, which is formed to execute the steps of the method according to the first aspect of the invention. The treatment device according to the invention allows that the disadvantages occurring in the use of usual ablative treatment devices, namely relatively long treatment times and a relatively high energy input by the laser into the cornea, are reliably avoided. These advantages are in particular achieved by the formation of the eye surgical laser as a photodisruptive laser.

Therein, the laser is suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz.

In further advantageous configurations of the treatment device according to the invention, the control device includes at least one storage device for at least temporary storage of at least one control dataset, wherein the control dataset or datasets include control data for positioning and/or for focusing individual laser pulses in the cornea; and comprises at least one beam device for beam guidance and/or beam shaping and/or beam deflection and/or beam focusing of a laser beam of the laser. Therein, the mentioned control datasets are usually generated based on a measured topography and/or pachymetry and/or morphology of the cornea to be treated and the type of the pathologically and/or unnaturally altered area to be removed within the cornea.

Further features and the advantages thereof can be taken from the descriptions of the first inventive aspect, wherein advantageous configurations of each inventive aspect are to be regarded as advantageous configurations of the respectively other inventive aspect.

A third aspect of the invention relates to a computer program including commands, which cause the treatment device according to the second inventive aspect to execute the method steps according to the first inventive aspect. A fourth aspect of the invention relates to a computer-readable medium, on which the computer program according to the third inventive aspect is stored. Further features and the advantages thereof can be taken from the descriptions of the first and the second inventive aspect, wherein advantageous configurations of each inventive aspect are to be regarded as advantageous configurations of the respectively other inventive aspect.

In addition, the invention relates to a method for separating a volume body with predefined interfaces from a human or animal cornea, comprising: controlling a laser by means of a control device such that it emits pulsed laser pulses in a predefined pattern into the cornea, wherein the interfaces of the volume body to be separated are defined by the predefined pattern and the interfaces are generated by means of photodisruption, wherein the interfaces of the volume body are determined such that a pathological and/or unnaturally altered area within the stroma of the cornea is enclosed, wherein the laser is controlled such that at least one incision or at least one aperture is generated in the cornea at a predefined angle and with a predefined geometry, wherein the incision or the aperture intersects an interface of the volume body and is formed up to a surface of the cornea such that the volume body is removable from the cornea via the incision or the aperture, and wherein the surface of the cornea is a surface of the eye artificially generated by means of ablation or displacement of an uppermost corneal layer and/or by means of production of a corneal flap.

The method according to the invention is usable for a plurality of methods in the correction of visual disorders of the eye. In particular, in the photorefractive keratectomy (PRK), in the epithelial laser-assisted keratomileusis (LASIK), the epithelial laser-assisted in-situ keratomileusis (Epi-LASIK) or the transepithelial photorefractive keratectomy (Trans-PRK), the method according to the invention can be used. It is a method, in which a tissue ablation occurs below an artificially generated corneal surface among other things. In contrast to the known method for tissue ablation of the artificial corneal surface, however, the laser is controlled such that a full-area ablation of a predefined volume body of the cornea is not effected in the method according to the invention, but the volume body is defined by the mentioned interfaces and the interfaces located in the cornea are generated by means of photodisruption. Therein, the volume body can be lenticularly formed.

In addition, the laser can be controlled such that the predefined pattern is at least partially circularly and/or spirally ablated. There is also the possibility that the predefined pattern is defined based on one or more control datasets, wherein the control dataset or datasets include control data for positioning and/or for focusing individual laser pulses in the cornea. The control datasets are generated at least by providing topographic and/or pachymetric and/or morphologic data of the untreated cornea and providing topographic and/or pachymetric and/or morphologic data of the pathologically altered area to be removed within the cornea.

Furthermore, the volume body to be separated can be formed such that a correction of a visual disorder of the eye is additionally effected by its removal. The pathologically altered area within the cornea can be an opacity and/or a scar.

Furthermore, there is the possibility that the control device is formed such that the laser emits laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz.

Further features and the advantages thereof can be taken from the descriptions of the first inventive aspect, wherein advantageous configurations of each inventive aspect are to be regarded as advantageous configurations of the respectively other inventive aspect.

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

### BRIEF DESCRIPTION OF DRAWINGS

FIG. 1 is a schematic representation of a treatment device according to the invention.

FIG. 2 is a schematic diagram of the generation of a volume body to be separated according to the first embodiment of the method according to the invention.

### DETAILED DESCRIPTION

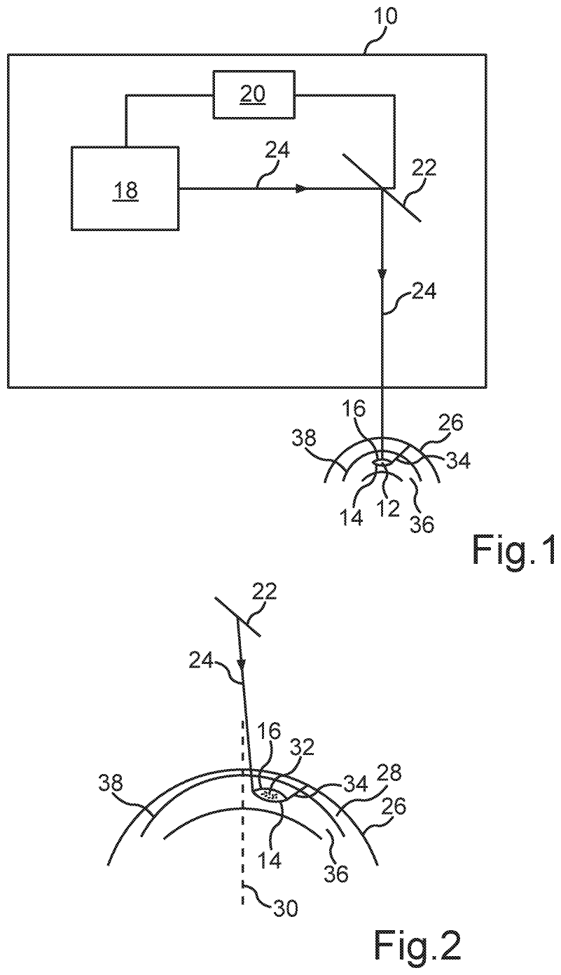

FIG. 1 shows a schematic representation of a treatment device 10 with an eye surgical laser 18 for the separation of a predefined corneal volume or volume body 12 with predefined interfaces 14, 16 of a cornea of a human or animal eye by means of photodisruption. One recognizes that a control device 20 for the laser 18 is formed besides the laser 18 such that it emits pulsed laser pulses in a predefined pattern into the cornea, wherein the interfaces 14, 16 of the volume body 12 to be separated are generated by means of photodisruption by the predefined pattern. In the illustrated embodiment, the interfaces 14, 16 form a lenticular volume body 12, wherein the position of the volume body 12 is selected such that a pathological and/or unnaturally altered area 32 (see FIG. 2) within the stroma 36 of the cornea is enclosed. Furthermore, it is recognizable from FIG. 1, that the so-called Bowman's membrane 38 is formed between the stroma 36 and the epithelium 28.

Furthermore, one recognizes that the laser beam 24 generated by the laser 18 is deflected in the direction of a surface 26 of the cornea by means of a beam device 22, namely a beam deflection device, such as for example a scanner. The beam deflection device 22 is also controlled by the control device 20 to generate the mentioned predefined pattern in the cornea.

The illustrated laser 18 is a photodisruptive laser, which is formed to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz.

In addition, the control device 20 comprises a storage device (not illustrated) for at least temporary storage of at least one control dataset, wherein the control dataset or datasets include control data for positioning and/or for focusing individual laser pulses in the cornea. The position data and/or focusing data of the individual laser pulses are generated based on a previously measured topography and/or pachymetry and/or the morphology of the cornea and the pathological and/or unnaturally altered area 32 to be removed within the stroma 36 of the eye.

FIG. 2 shows a schematic diagram of the generation of the volume body 12 to be separated according to an embodiment of the present method. One recognizes that the interfaces 14, 16 are generated by means of the pulsed laser beam 24, which is directed in the direction of the cornea or in the direction of the surface 26 of the cornea via the beam deflection device 22. Therein, the interfaces 14, 16 form a lenticular volume body, which encloses the pathological and/or unnaturally altered area 32 within the stroma 36. Furthermore, the laser 18 generates a further incision 34 in the illustrated embodiment, which intersects the volume body at a predefined angle and with a predefined geometry and is formed up to the surface 26 of the cornea. The volume body defined by the interfaces 14, 16 can then be removed from the cornea via the incision 34. In the illustrated embodiment, the pathological and/or unnaturally altered area 32 is formed within the stroma 36 and outside of an optical axis 30 of the eye.

In the illustrated embodiment, the interface 14, that is the interface located deeper in the eye or the stroma 36, is first formed by means of the laser beam 24. This can be effected by at least partially circularly and/or spirally guiding the laser beam 24 according to the predefined pattern. Subsequently, the interface 16 is generated in comparable manner such that the interfaces 14, 16 form the lenticular volume body 12 (see also FIG. 1). Subsequently, the incision 34 is also generated by the laser 18. However, the order of the generation of the interfaces 14, 16 and the incision 34 can also be changed.

1. A method for controlling an eye surgical laser for the separation of a volume body with predefined interfaces from a human or animal cornea, comprising:
controlling the laser by means of a control device such that it emits pulsed laser pulses in a predefined pattern into the cornea, wherein the interfaces of the volume body to be separated are defined by the predefined pattern wherein the interfaces are generated by means of photodisruption, and wherein the interfaces of the volume body are determined such that a pathological and/or unnaturally altered area within the stroma of the cornea is enclosed,
wherein said laser is controlled such that at least one incision or at least one aperture is generated in the cornea at a predefined angle and with a predefined geometry, wherein said incision or said aperture intersects an interface of the volume body and is formed up to a surface of the cornea, such that the volume body is removable from the cornea via said incision or said aperture, and
wherein the surface of the cornea is a surface of the eye artificially generated by means of ablation or displacement of an uppermost corneal layer and/or by means of production of a corneal flap.

2. The method according to claim 1, wherein characterized in that the volume body is lenticularly formed.

3. The method according to claim 1, wherein said laser is controlled such that the predefined pattern is at least partially circularly and/or spirally ablated.

4. The method according to claim 1, wherein the predefined pattern is defined based on one or more control datasets, wherein the control dataset or datasets include control data for positioning and/or for focusing individual laser pulses in the cornea.

5. The method according to claim 4, wherein the control datasets are generated at least by providing topographic and/or pachymetric and/or morphologic data of the untreated cornea and providing topographic and/or pachymetric and/or morphologic data of the pathologically altered area to be removed within the cornea.

6. The method according to claim 1, wherein said volume body to be separated is furthermore formed such that a correction of a visual disorder of the eye is additionally effected by the removal of the volume body.

7. The method according to claim 1, wherein said pathologically altered area within the cornea is an opacity and/or a scar.

8. The method according to claim 1, wherein said control device is formed such that the laser emits laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz.

9. A treatment device with at least one eye surgical laser for the separation of a corneal volume with predefined interfaces from a human or animal eye by means of photodisruption and at least one control device for the laser or lasers, which is formed to execute the steps of the method according to claim 1.

10. The treatment device according to claim 9, wherein said laser is suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz.

11. The treatment device according to claim 9, wherein said control device
comprises at least one storage device for at least temporary storage of at least one control dataset, wherein the control dataset or datasets include control data for positioning and/or for focusing individual laser pulses in the cornea; and
comprises at least one beam device for beam guidance and/or beam shaping and/or beam deflection and/or beam focusing of a laser beam of the laser.

12. A computer program including commands, which cause a treatment device with at least one eye surgical laser for the separation of a corneal volume with predefined interfaces from a human or animal eye by means of photodisruption and at least one control device for the laser or lasers to execute the method steps according to claim 1.

13. A computer-readable medium, on which the computer program according to claim 12 is stored.

14. A method for separating a volume body with predefined interfaces from a human or animal cornea, comprising:
controlling a laser by means of a control device such that it emits pulsed laser pulses in a predefined pattern into the cornea, wherein the interfaces of the volume body to be separated are defined by the predefined pattern and the interfaces are generated by means of photodisruption, wherein the interfaces of the volume body are determined such that a pathological and/or unnaturally altered area within the stroma of the cornea is enclosed,
wherein the laser is controlled such that at least one incision or at least one aperture is generated in the cornea at a predefined angle and with a predefined geometry, wherein the incision or the aperture intersects an interface of the volume body and is formed up to a surface of the cornea, such that the volume body is removable from the cornea via the incision or the aperture, and
wherein the surface of the cornea is a surface of the eye artificially generated by means of ablation or displacement of an uppermost corneal layer and/or by means of production of a corneal flap.

15. The method according to claim 14, wherein said volume body is lenticularly formed.

16. The method according to claim 14, wherein said laser is controlled such that the predefined pattern is at least partially circularly and/or spirally ablated.

17. The method according to claim 14, wherein said predefined pattern is defined based on one or more control datasets, wherein the control dataset or datasets include control data for positioning and/or for focusing individual laser pulses in the cornea.

18. The method according to claim 17, wherein said control datasets are generated at least by providing topographic and/or pachymetric and/or morphologic data of the untreated cornea and providing topographic and/or pachymetric and/or morphologic data of the pathologically altered area to be removed within the cornea.

19. The method according to claim 14, wherein said volume body to be separated is furthermore formed such that a correction of a visual disorder of the eye is additionally effected by the removal of the volume body.

20. The method according to claim 14, wherein said pathologically altered area within the cornea is an opacity and/or a scar.

21. The method according to claim 14, wherein said control device is formed such that the laser emits laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 KHz, preferably between 100 KHz and 100 MHz.
